(19) United States
(12) Patent Application Publication
(10) Pub. No.: US 2006/0195068 A1
(43) Pub. Date: Aug. 31, 2006

(54) Disposable garment having moisture indicator viewable through inspection window

(76) Inventor: Juliette E. Lawando, Schaumburg, IL (US)

Correspondence Address: FACTOR & LAKE, LTD, 1327 W. WASHINGTON BLVD., SUITE 5G/H, CHICAGO, IL, 60607, US

(21) Appl. No.: 11/351,340

(22) Filed: Feb. 9, 2006

Related U.S. Application Data
(60) Provisional application No. 60/593,751, filed on Feb. 10, 2005.

(51) Int. Cl. A61F 13/15

(52) U.S. Cl. 604/361

(57) ABSTRACT

A disposable garment is disclosed with a flexible inspection window on the outer portion of the garment to enable a caretaker to easily determine whether the garment is soiled.

### FEDERALLY SPONSORED RESEARCH OR DEVELOPMENT

None.

### RELATED APPLICATIONS

This application claims the benefit of U.S. Provisional Patent Application Ser. No. 60/593,751, filed on Feb. 10, 2005, and incorporated herein by reference.

This application also incorporates by reference U.S. Pat. No. 6,213,992, issued to Dreier on Apr. 10, 2001, and U.S. Pat. No. 5,078,708, issued to Haque on Jan. 7, 1992.

### FIELD OF THE INVENTION

The present invention relates to disposable, absorbent garments and, more particularly, to a disposable garment in which a flexible inspection window is provided on the outer portion of the garment, in addition, a moisture indicator is provided to enable a caretaker to easily determine whether the garment is soiled.

### BACKGROUND OF THE INVENTION

Many times, especially with infants, it is difficult to determine whether a diaper is soiled by urine without removing the diaper or tampering with it. The present invention provides for easily determining whether the diaper is soiled without removing or tampering with the diaper.

### SUMMARY OF INVENTION

In an embodiment, the present invention is directed to a disposable, absorbent garment such as a diaper having a flexible inspection window and a moisture indicator to enable a caretaker to easily determine whether the diaper is soiled.

In another embodiment, the present invention is directed to a disposable, absorbent garment such as a diaper having a flexible inspection window to enable a caretaker to easily determine whether the diaper is soiled.

Other embodiments, systems, methods, features, and advantages of the present invention will be, or will become, apparent to one having ordinary skill in the art upon examination of the following drawings and detailed description. It is intended that all such additional systems, methods, features, and advantages included within this description be within the scope of the present invention, and be protected by the accompanying claims.

### DESCRIPTION OF THE DRAWINGS

The invention may be better understood with reference to the following drawings. The components in the drawings are not necessarily to scale, emphasis instead being placed upon illustrating the principles of the invention. In the drawings, like reference numerals designate corresponding parts throughout the several views.

FIG. 1 is a front view of a garment in accordance with the present invention with tabs secured to the front of the garment;

FIG. 2 is a perspective view of the garment of FIG. 1;

FIG. 3 is a perspective view of an alternative embodiment of a garment in accordance with the present invention; and,

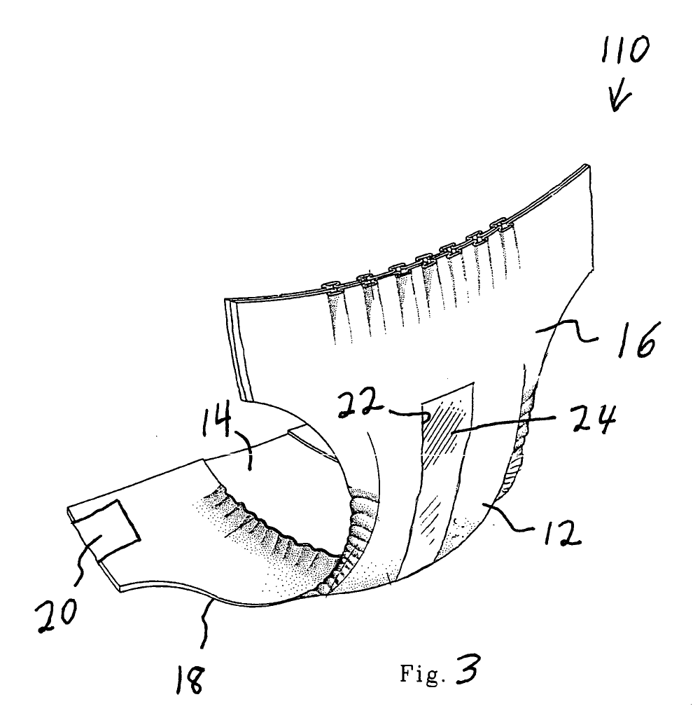

FIG. 4 is a greatly enlarged partial cross sectional view about a window of yet another embodiment of a garment similar to that of FIG. 1.

### DESCRIPTION OF DETAILED EMBODIMENTS

The following descriptions of detailed embodiments are for exemplifying the principles and advantages of the inventions claimed herein. They are not to be taken in any way as limitations on the scope of the inventions.

In the FIGURES, the following reference numbers are used to depict corresponding elements wherein reference numbers having the same last two digits correspond to like elements:

| Reference Number | Element |
|---|---|
| 10 | garment (e.g., diaper) |
| 12 | outer surface |
| 14 | inner surface |
| 16 | front |
| 18 | back |
| 20 | tabs |
| 22 | window opening |
| 24 | window covering |
| 26 | absorbent material |
| 28 | compartment |
| 30 | indicator material having color changeable dye |
| | indicator |
| 32 | semi-permeable layer |

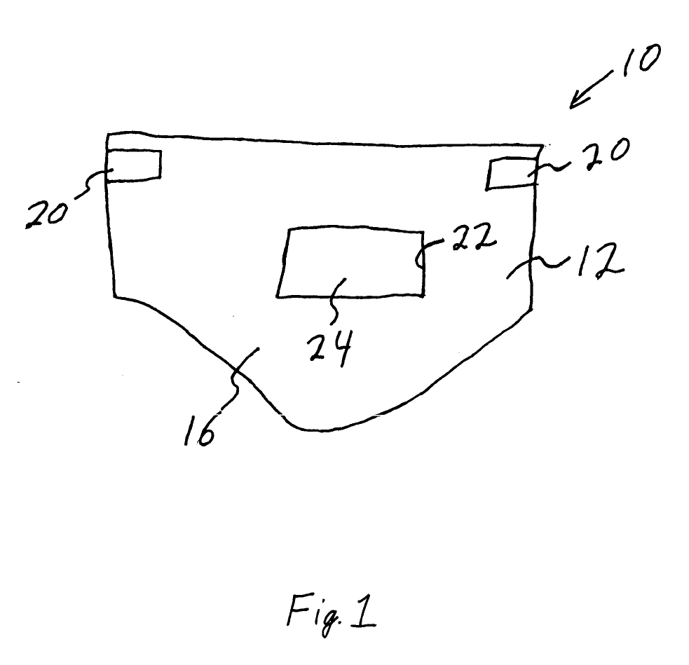

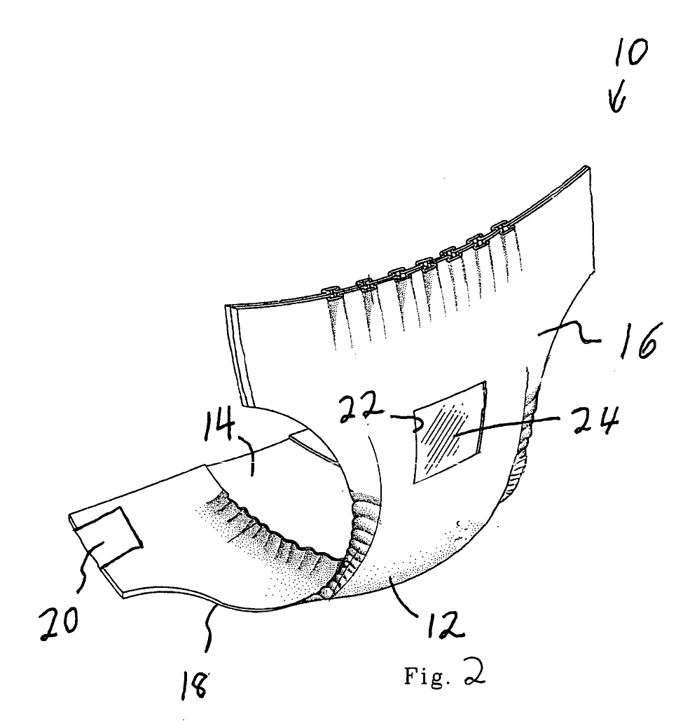

Turning to FIGS. 1 and 2, a garment 10 is depicted in accordance with the present invention. The garment 10 is preferably a diaper having an outer surface 12 comprising a layer of material, and an inner surface 14, comprising a layer of material. Located between at least a portion of the outer surface 12 and the inner surface 14 is a layer or clump of conventional absorbent material or materials. The diaper 10 also includes a front 16, a back 18, and conventional attachment means, such as flaps or tabs 16, for securing the diaper to a person in a conventional manner. In an embodiment, a suitable like garment that can be modified to incorporate the features of the present invention is currently sold as PAMPERS CRUISERS.

The front 16 of the garment 10 has an opening or aperture 22 that is covered by a flexible transparent material 24 that allows for a person to visually observe the color of the absorbent material. In an embodiment, the transparent window 24 is a clear plastic sheet attached to the outer layer 12 of the diaper by conventional means such as, but not limited to, gluing or the like. Preferably, the window is attached in a manner that prevents urine or the like from leaking outside the diaper.

As will be appreciated by those having ordinary skill in the art, the opening 22 can have a variety of shapes including, but not limited to, a square, a rectangular, a circular, an oval, or the like. As stated previously, the window opening 22 can be located on the front of the diaper 10. However, in alternative embodiments, the window can be provided on the back of the diaper, or the diaper can have windows on the front and back of the diaper 10, or a single window can extend from the front to the back of the diaper.

In an embodiment, but not necessarily, the window opening 22 is about one (1) and one-half (½) inches wide, and located about five (5) inches from the top of the diaper.

In an embodiment, the absorbent material within the diaper is white or some other color other than yellow or yellowish. Accordingly, when the absorbent material absorbs urine, the absorbent material will be discolored, and thus a caregiver with be alerted that the garment has become soiled when seeing the discoloration of the absorbent material through the window.

In yet another embodiment, a portion of the absorbent material about, adjacent, or abutting the window 24 can be treated with a water-soluble dye or other conventional substance or indicator that will change color when exposed to moisture. In an embodiment, the indicator or substance is preferably colorless when it is dry, and changes to a humanly visible color such as, but not limited to, red, blue, yellow, or another color when the substance is exposed to moisture, and in particular, urine. Accordingly, this embodiment aids in rapidly determining, via the window 24, if the diaper has become soiled. This is especially useful if the urine has very little color.

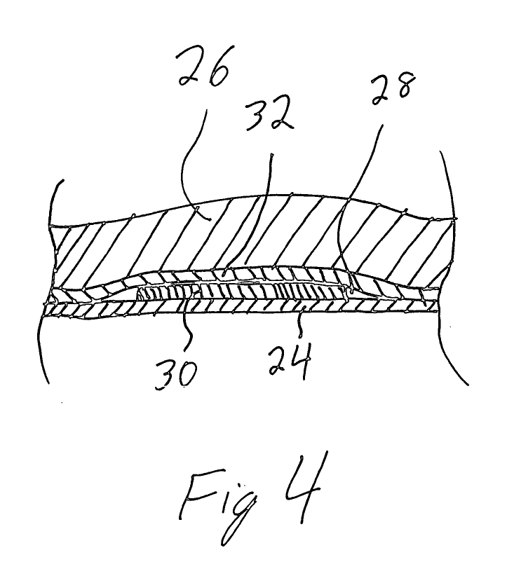

In a further embodiment, as illustrated in FIG. 4, a compartment 28 can be provided to contain a material or indicator 30 having a chemical compound such as a color changeable dye. For instance, the indicator 30 can be, but is not necessarily limited to, a chemical compound pH indicator such as litmus that is absorbed onto filter paper (i.e., litmus paper). Suitable indicators include, but are not limited to, Congo Red (i.e., the sodium salt of benzidinediazo-bis-1-naptylamine-4-sulfonic acid), which has a blue color at low pH, and a red color at high pH. Thus, the indicator will turn red when exposed to urine having a pH of about 5.5 to 6.5.

To prevent contact of the chemical compound with the baby when the compound becomes wet, the compartment 28 is preferably has a layer 32 placed over the material 30 at attached to the window 24. In an embodiment, the entire outer periphery of the layer 32 is attached to the window 24 by conventional means such as by gluing, ultrasonic welding, or other conventional means. Further, the layer 32 can be a conventional semipermeable membrane that will allow urine into the compartment 28, but will not allow the chemical compound on the material 28 to escape the compartment. Such semipermeable membranes are well know to those having ordinary skill in the art, and are thus not explained further herein as the semipermenable membrane, by itself, is not being claimed as the invention.

It should be emphasized that the above-described embodiments of the present invention, particularly, any “preferred” embodiments, are possible examples of implementations merely set forth for a clear understanding of the principles of the invention. Many variations and modifications may be made to the above-described embodiment(s) of the invention without substantially departing from the spirit and pnrinciples of the invention. All such modifications are intended to be included herein within the scope of this disclosure and the present invention, and protected by the following claims.

What is claimed is:

1. An apparatus comprising:
an outer layer of material having a opening;
a transparent material attached to the outer layer of material over the opening;
an inner layer of material;
an absorbent material between at least a portion of the outer and inner layers; and, wherein at least a portion of the absorbent material is visible through the transparent material.

2. The apparatus of claim 1 further comprising an indicator that changes color when exposed to urine and visible via the transparent material.

3. The apparatus of claim 1 wherein the outer layer is liquid impermeable.

4. The apparatus of claim 1 wherein the inner layer is liquid permeable.

5. The apparatus of claim 1 wherein the transparent material is liquid impermeable.

6. The apparatus of claim 1, wherein the transparent material is flexible plastic.

7. The apparatus of claim 1, wherein the outer layer has a front, with the opening located in the front.

8. The apparatus of claim 1, wherein the outer layer has a back, with the opening located in the back.

9. The apparatus of claim 1, wherein the outer layer has a front and a back, with the opening located in the front and a second opening located in the back, and another at least partially transparent material attached to the outer layer of material over the second opening.

10. The apparatus of claim 1, wherein the outer layer has a front and a back, with the opening extending from the front to the back.

11. The apparatus of claim 9, wherein the opening extends over a crotch region between the front and the back.

12. The apparatus of claim 9, wherein the transparent material is a plastic elongated strip.

13. The apparatus of claim 2, wherein the indicator is within a compartment.

14. The apparatus of claim 2, wherein the indicator is between a semipermeable layer and the transparent material.

15. The apparatus of claim 2, wherein the indicator is within the absorbent material.

16. An apparatus comprising:
an outer layer of material having a opening;
an at least partially transparent material attached to the outer layer of material over the opening;
an inner layer of material;
an absorbent material between at least a portion of the outer and inner layers; and, wherein at least a portion of the absorbent material is visible through the transparent material.

17. The apparatus of claim 16 further comprising an indicator that changes color when exposed to urine and visible via the transparent material.

18. An apparatus comprising:
an outer layer of material having a opening;
an at least partially transparent material attached to the outer layer of material over the opening; and,
an inner layer of absorbent material wherein at least a portion of the absorbent material is visible through the transparent material.

19. The apparatus of claim 18 further comprising an indicator that changes color when exposed to urine and visible via the transparent material.

20. The apparatus of claim 19, wherein the outer layer has a front and a back, with the opening extending from the front to the back.
